(19) United States
(12) Patent Application Publication
(10) Pub. No.: US 2021/0024459 A1
(43) Pub. Date: Jan. 28, 2021

(54) METHOD FOR RECOVERING DIISOCYANATES FROM DISTILLATION RESIDUES

(71) Applicant: Covestro Deutschland AG, Leverkusen (DE)

(72) Inventors: Joerg Morawski, Leverkusen (DE); Tim Loddenkemper, Dormagen (DE); Stefanie Lang, Leverkusen (DE); Rainer Bellinghausen, Odenthal (DE); Ulrich Liesenfelder, Bergisch Gladbach (DE); Michael Merkel, Düsseldorf (DE); Marc Seekamp, Köln (DE); Ariane Dunker, Köln (DE)

(21) Appl. No.: 17/043,117

(22) PCT Filed: Apr. 3, 2019
(86) PCT No.: PCT/EP2019/058438

§ 371 Date: Sep. 29, 2020

(30) Foreign Application Priority Data
Apr. 6, 2018 (EP) 18166140.6

(51) Int. Cl. C07C 263/20; F26B 11/12

(52) U.S. Cl. CPC C07C 263/20; C07C 265/12; F26B 11/12

(57) ABSTRACT

The invention relates to a method for recovering monomer diisocyanates, which are solid at room temperature, from a distillation residue, said method comprising the following steps: (i) preparing at least one residue which contains diisocyanates, which are solid at room temperature, and (ii) separating the residue in at least one kneader-dryer, paddle-dryer and/or roller-dryer in the presence of less than 2 wt % bitumen, based on the mass of the residue prepared in step (i), into a gaseous portion, containing monomer diisocyanate that is solid at room temperature, and a brittle residue depleted of diisocyanate, which is solid at room temperature.

The present invention relates to a process for recovering monomeric, room temperature solid diisocyanates from a distillation residue. The invention further relates to a monomeric, room temperature solid diisocyanate obtainable by this process and to the use of a kneader-drier, paddle drier or drum drier for embrittlement of a residue containing room temperature solid diisocyanates.

The invention also relates to a composition comprising at least one monomeric, room temperature solid diisocyanate of the invention, and elastomers obtainable from said composition.

The industrial scale preparation of diisocyanates by reacting amines with phosgene in solvents is known and described in detail in the literature (Ullmanns Encyklopadie der technischen Chemie [Ullmann's Encyclopedia of Industrial Chemistry], 4th edition, volume 13, pages 347-357, Verlag Chemie, GmbH, D-6940 Weinheim, 1977 or else EP 1 575 908 A1). Usually, the production of pure distilled diisocyanates in the distillation processes affords a by-product stream that has to be disposed of as residue after widely possible distillative removal of free isocyanates. Depending on the chemical nature of the diisocyanate prepared, this residue contains a considerable proportion of monomeric diisocyanate. It was thus desirable to be able to recover the monomeric diisocyanate to the best possible degree from the residue in order to be able to increase the overall yield without worsening the overall economic viability of the process.

WO 2007/036479 A1 discloses a general process for purifying isocyanate-containing residues, in which it is less preferably possible also to use naphthyl diisocyanate. No working examples are disclosed. The distillation takes place at temperatures between 210° C. and 330° C., forming a high-viscosity liquid and/or a non-embrittling, i.e. pasty, solid, which is free-flowing under the conditions that exist in the apparatus. According to the residue, under the process conditions mentioned, it is not possible to rule out oligomerization of the isocyanates present, and so there is ultimately the risk of embrittlement, for which the apparatuses are not designed.

EP 0 626 368 A1 describes a process for preparing pure distilled isocyanates in which the residue is admixed with 2% to 50% by weight of high-boiling hydrocarbons and the isocyanate is extracted from this residue at temperatures of 160° C. to 280° C. Kneader-driers are used in this process, which mean relatively complex technology with mechanically moving parts. Since the continuous discharge of free-flowing material has to be assured in the case of this technology, the nature of the residue is crucial and greatly restricts utilization. Another disadvantage is the handling and disposal of relatively large amounts of an auxiliary otherwise extraneous to the process.

U.S. Pat. No. 3,694,323 discloses a process for recovering an isocyanate from its phosgenation residue with the aid of what is called an isocyanate exchange medium which has a higher boiling point than the isocyanate to be purified and hence lowers the viscosity of the phosgenation residue and enables purification. What is disadvantageous about this process, however, is that the purified isocyanate is contaminated with the isocyanate exchange medium as was already the case in EP 0 626 368 A1, additional work for the handling and disposal of the isocyanate exchange medium.

DE10260092A1 describes a process for purifying isocyanates, wherein a residue stream containing unevaporable residue and isocyanate is obtained in a distillation step. This is separated in a step c) into a further, isocyanate-containing vapor stream and a stream containing essentially unevaporable residue. The further properties of this residue are described as being highly viscous or solid, but without stating the conditions under which this is the case. It can be concluded from the selection of recommended drying apparatuses and the working example using tolylene diisocyanate that there must be a free-flowing, viscous or highly viscous phase in the drier since most of the apparatuses mentioned are unsuitable for the occurrence of solids.

In the prior art cited, the removal is thus effected either with the aid of relatively large amounts of additional agents that enable embrittlement of the residue down to a free-flowing residue or by cooling the residue even in the drying apparatus with the disadvantages described above. Alternatively, the separation is effected under such conditions that the residue remains at least pasty and conveyable.

It has now been found that, surprisingly, distillation residues of room temperature solid diisocyanates are particularly suitable for separation, even in the absence of bitumen, in a kneader-drier, paddle drier or drum drier, into a gaseous, monomeric diisocyanate-containing portion and a brittle residue depleted of monomeric, room temperature solid diisocyanate.

The invention therefore provides a process for recovering monomeric, room temperature solid diisocyanates from a distillation residue, comprising the following steps:
(i) providing at least one residue containing room temperature solid diisocyanates and
(ii) separating the residue in a kneader-drier, paddle drier and/or drum drier in the presence of less than 2% by weight of bitumen, based on the mass of the residue provided in step (i), into a gaseous portion containing monomeric, room temperature solid diisocyanate, and a brittle residue depleted of monomeric, room temperature solid diisocyanate.

In the present context, the term “brittle” is used for the identification of substances, for example of the residue depleted of monomeric, room temperature solid diisocyanate, when the substances, in the stress-strain diagram obtained when a sample is subjected to a tensile force F and the resultant change in length ΔL is plotted against it, have a steep straight line as per Hooke's law that characterizes the proportional range of stress and strain, and the Hooke's straight line ends with fracture.

In the present context, “room temperature” is understood to mean a temperature of 25° C.

In a preferred embodiment of the process of the invention, the kneader-drier, paddle drier or drum drier is designed without a cooling zone. In this way, internal heat losses are avoided and the probability of leaks from the respective drier is reduced.

In the present context, the term “cooling zone” is understood to mean that the drier includes a region in which the wall temperature can be regulated to a lower temperature independently of the rest of the drier. This is intended to assist the solidification of the residue, but is associated with the abovementioned disadvantages.

In a further preferred embodiment of the process, the residue is separated in step (ii) in a kneader-drier or a drum drier and more preferably in a drum drier. These are particularly suitable for processing embrittling mixtures. Particularly drum driers have the advantage that embrittlement proceeds within a very short time owing to their construction. In principle, the embrittlement is based essentially on two effects: the withdrawal of diisocyanate by evaporation and the oligomerization of diisocyanate. Specifically in the case of drum driers, the contribution of the evaporation of diisocyanate is elevated compared to other driers, which leads to a higher yield of the material of value.

In a particularly preferred embodiment of the process, the separation in step (ii) is effected in the presence of ≤1% by weight of bitumen, preferably in the absence of bitumen.

In a further preferred embodiment of the process, the temperature in step (ii) is ≥130° C. to ≤270° C., preferably ≥140° C. to ≤200° C. and more preferably ≥150° C. to ≤190° C. Within this temperature range, there is firstly evaporation or sublimation of monomeric diisocyanate out of the residue, such that it can be recovered. There is secondly already a limited degree of oligomerization of the diisocyanates, which contributes to the desired embrittlement of the residue without resulting in prohibitive yield losses.

In a further preferred embodiment of the, the pressure in step (ii) is ≥0.1 mbar to ≤1020 mbar, preferably ≥0.5 mbar to ≤25 mbar and more preferably ≥1 mbar to ≤10 mbar. This preferred pressure range supports rapid removal of the monomeric diisocyanate, such that the dwell times in the corresponding drier can be kept short. Considerably lower pressures are not beneficial since these make it difficult to condense the portion obtained in gaseous form in step (ii) for further use.

Suitable room temperature solid diisocyanates are preferably naphthalene 1,5-diisocyanate, naphthalene 1,8-diisocyanate, phenylene 1,4-diisocyanate, tetralin diisocyanate, o-toluidine diisocyanate, durene diisocyanate, benzidine diisocyanate and/or anthrylene 1,4-diisocyanate.

In a further preferred embodiment of the process, the room temperature solid diisocyanate is preferably naphthalene 1,5-diisocyanate, naphthalene 1,8-diisocyanate or phenylene 1,4-diisocyanate, more preferably naphthalene 1,5-diisocyanate or phenylene 1,4-diisocyanate, and most preferably naphthalene 1,5-diisocyanate. It is a feature of these isocyanates that they have elevated reactivity, which contributes to reliable embrittlement of the residue.

Suitable kneader-driers, paddle driers or drum driers are especially those driers that have devices for the cleaning of the moving parts, and of the drier housing. This is especially true of the heated surfaces. More preferably, scraper blades and/or opposing hooks serve to detach the embrittled residue.

Suitable examples are vacuum drum driers with one drum or preferably twin-drum vacuum driers. These are what are called thin-layer driers, in which the material to be dried is applied to slow-rotating, usually steam-heated drums. In the course of this, fractions of the material to be dried evaporate and the remaining residue is scraped off the drum(s). Owing to the high surface area for conductive heat transfer, quite short and hence gentle dwell times for drying are sufficient. In the case of operation under reduced pressure, the unevaporated residue is either discharged via corresponding vacuum locks or operation is effected batchwise, in which case the drum of the drier is intermittently brought to atmospheric pressure in order to discharge the dried material.

Suitable kneader-driers are, for example, what are called single- or twin-shaft kneader reactors having large heating surfaces and tools for kneading and mixing the material to be dried. The kneading tools are arranged here on the shaft(s) of the drier such that they ensure firstly radial mixing and secondly axial transport of the material being dried. Simultaneously, in the case of twin-shaft apparatuses, the shafts can be operated in a co-rotating or else counter-rotating manner as required. The gap sizes are preferably chosen such that the heated surfaces are cleaned automatically by the movement of the kneading tools. The intensive mixing leads to rapid evaporation of the evaporable fractions and to additional energy input into the material to be dried.

Suitable paddle driers consist, for example, of a heated housing in which there are one or more, usually likewise heated shafts. Paddles are arranged on the shafts in such a way that the material to be dried is in turn mixed radially and simultaneously conveyed axially. This is achieved, for example, by an oblique position of the paddles, such that a change in the direction of rotation also achieves a change in the axial conveying direction, which can be utilized for discharge of the dried material.

Preferably, the average dwell time of the residue in the kneader-drier, paddle drier or drum drier is from ≥0.5 minute to ≤4 hours, more preferably from ≥0.5 minute to ≤60 minutes, even more preferably from ≥0.5 minute to ≤15 minutes and most preferably from ≥1 minute to ≤10 minutes.

The dwell time ultimately required, which is a compromise between reliable embrittlement, high recovery of monomeric diisocyanate and limited apparatus size, depends here on the chosen isocyanate, the pressure and the temperature in the drier, and can be ascertained by the person skilled in the art in a test series.

The residue detached can preferably be discharged semicontinuously, i.e. in cycles, and then cooled down. In a particularly preferred embodiment, the discharge from the apparatus is via a vacuum lock.

In a further preferred embodiment of the process, the conditions in step (ii) are chosen such that the brittle residue obtained in step (ii) contains ≤5% by weight, preferably ≤2% by weight, more preferably ≤0.2% by weight and most preferably <0.1% by weight of monomeric, room temperature solid diisocyanate based on the total mass of the residue. This is particularly advantageous since higher concentrations of monomeric diisocyanate firstly mean a loss of material of value; secondly, the monomeric diisocyanates can in some cases be harmful to health or have corrosive properties.

In principle, the room temperature solid diisocyanates can be prepared by any routes, for example by reaction of the corresponding diamines or salts thereof with phosgene. All that is important in each case is that the process utilized leaves at least one residue containing room temperature solid diisocyanates that can then be used in step (i) of the invention.

In a further preferred embodiment of the process, the residue containing room temperature solid diisocyanates that is provided in step (i) comes from the distillation of a diisocyanate prepared by phosgenating the corresponding diamines More preferably, the residue containing room temperature solid diisocyanates which is provided in step (i) comes from the phosgenation of the corresponding diamines, preferably from the liquid phase phosgenation of the corresponding diamines.

In the present context, the term “corresponding diamine” is in each case understood to mean the room temperature solid diisocyanate that is to be prepared in which the two isocyanate groups have been exchanged for amino groups. By way of example, naphthalene 1,5-diamine is the corresponding diamine of naphthalene 1,5-diisocyanate. When the room temperature solid diisocyanate is an isomer mixture, a corresponding isomer mixture of diamines is used.

As apparent from the process of the invention, kneader-driers, paddle driers and/or drum driers can be used very efficiently for depletion of monomeric, room temperature solid diisocyanates from a residue containing room temperature solid diisocyanates and/or embrittlement of a residue containing room temperature solid diisocyanates.

The invention further provides a monomeric, room temperature solid diisocyanate obtained or obtainable by the process of the invention. By very substantially dispensing with auxiliaries such as bitumen in step (ii) of the process, contamination of the monomeric, room temperature solid diisocyanate that is recovered from the residue with these auxiliaries is also minimized or even avoided entirely.

The invention also further provides for the use of a kneader-drier, paddle drier and/or drum drier for minimizing the use of auxiliaries in the embrittlement of a residue containing room temperature solid diisocyanates. In the case of this use, the residue containing room temperature solid diisocyanates is preferably a distillation residue.

The monomeric, room temperature solid diisocyanates obtained by the process of the invention can be sent to various end uses. Particular mention should be made of further processing with NCO-reactive compounds such as polyols to give polyurethanes, optionally via prepolymers as intermediates.

These polyurethanes preferably have apparent densities of 200 kg/m³ to 1400 kg/m³, more preferably of 600 kg/m³ to 1400 kg/m³ and most preferably of 800 kg/m³ to 1400 kg/m³. Very particular preference is given to producing cellular or bulk cast elastomers, more preferably polyester polyol-based cast elastomers.

The present invention thus further provides a composition comprising at least monomeric, room temperature solid diisocyanate of the invention and at least one NCO-reactive compound, preferably at least one polyester polyol.

The composition may additionally comprise customary assistants and additives, for example rheology improvers (for example ethylene carbonate, propylene carbonate, dibasic esters, citric esters), stabilizers (for example Bronsted and Lewis acids, for instance hydrochloric acid, phosphoric acid, benzoyl chloride, organo mineral acids such as dibutyl phosphate, and also adipic acid, malic acid, succinic acid, pyruvic acid or citric acid), UV stabilizers (for example 2,6-dibutyl-4-methylphenol), hydrolysis stabilizers (for example sterically hindered carbodiimides), emulsifiers and catalysts (for example trialkylamines, diazabicyclooctane, tin dioctoate, dibutyltin dilaurate, N-alkylmorpholine, lead octoate, zinc octoate, tin octoate, calcium octoate, magnesium octoate, the corresponding naphthenates and p-nitrophenoxide and/or else mercury phenylneodecanoate) and fillers (for example chalk), dyes which may be incorporable into the polyurethane/polyurea to be formed at a later stage (which thus possess Zerewitinoff-active hydrogen atoms) and/or color pigments.

NCO-reactive compounds used may be any compounds known to those skilled in the art.

As NCO-reactive compounds may polyether polyols, polyester polyols, polycarbonate polyols and polyether amines having an average OH or NH functionality of at least 1.5, and short-chain polyols and polyamines (chain extenders or crosslinkers), as are sufficiently well known from the prior art. These may be, for example, low molecular weight diols (e.g. 1,2-ethanediol, 1,3- or 1,2-propanediol, 1,4-butanediol), triols (e.g. glycerol, trimethylolpropane) and tetraols (e.g. pentaerythritol), but also higher molecular weight polyhydroxyl compounds such as polyether polyols, polyester polyols, polycarbonate polyols, polysiloxane polyols, polyamines and polyether polyamines and polybutadiene polyols.

Polyether polyols are obtainable in a manner known per se by alkoxylation of suitable starter molecules under base catalysis or by the use of double metal cyanide compounds (DMC compounds). Examples of suitable starter molecules for the preparation of polyether polyols are simple low molecular weight polyols, water, organic polyamines having at least two N-H bonds, or any mixtures of such starter molecules. Preferred starter molecules for preparation of polyether polyols by alkoxylation, especially by the DMC process, are especially simple polyols such as ethylene glycol, propylene 1,3-glycol and butane-1,4-diol, hexane-1,6-diol, neopentyl glycol, 2-ethylhexane-1,3-diol, glycerol, trimethylolpropane, pentaerythritol, and low molecular weight hydroxyl-containing esters of such polyols with dicarboxylic acids of the kind specified hereinafter by way of example, or low molecular weight ethoxylation or propoxylation products of such simple polyols, or any desired mixtures of such modified or unmodified alcohols. Alkylene oxides suitable for the alkoxylation are especially ethylene oxide and/or propylene oxide, which can be used in the alkoxylation in any sequence or else in a mixture.

Polyester polyols can prepare in a known manner by polycondensation of low molecular weight polycarboxylic acid derivatives, for example succinic acid, adipic acid, suberic acid, azelaic acid, sebacic acid, dodecanedioic acid, tetrahydrophthalic anhydride, hexahydrophthalic anhydride, tetrachlorophthalic anhydride, endomethylenetetrahydrophthalic anhydride, glutaric anhydride, maleic acid, maleic anhydride, fumaric acid, dimer fatty acid, trimer fatty acid, phthalic acid, phthalic anhydride, isophthalic acid, terephthalic acid, citric acid or trimellitic acid, with low molecular weight polyols, for example ethylene glycol, diethylene glycol, neopentyl glycol, hexanediol, butanediol, propylene glycol, glycerol, trimethylolpropane, 1,4-hydroxymethylcyclohexane, 2-methyl-1,3-propanediol, butane-1,2,4-triol, triethylene glycol, tetraethylene glycol, polyethylene glycol, dipropylene glycol, polypropylene glycol, dibutylene glycol and polybutylene glycol, or by ring-opening polymerization of cyclic carboxylic esters such as ϵ-caprolactone. It is moreover also possible to polycondense hydroxycarboxylic acid derivatives, for example lactic acid, cinnamic acid or w-hydroxycaproic acid to form polyester polyols. However, it is also possible to use polyester polyols of oleochemical origin. Such polyester polyols can be prepared, for example, by full ring-opening of epoxidized triglycerides of an at least partly olefinically unsaturated fatty acid-containing fat mixture with one or more alcohols having 1 to 12 carbon atoms and subsequent partial transesterification of the triglyceride derivatives to alkyl ester polyols having 1 to 12 carbon atoms in the alkyl radical.

The NCO-reactive compound may contain short-chain polyols or polyamines as crosslinker component or chain extender. Typical chain extenders are diethylenetoluenediamine (DETDA), 4,4′-methylenebis(2,6-diethyl)aniline (MDEA), 4,4′-methylenebis(2,6-diisopropyl)aniline (MDIPA), 4,4′-methylenebis(3-chloro-2,6-diethyl)aniline (MCDEA), dimethylthiotoluenediamine (DMTDA, Ethacure® 300), N,N′-di(sec-butyl)aminobiphenylmethane (DBMDA, Unilink® 4200) or N,N′-di-sec-butyl-p-phenylenediamine (Unilink® 4100), 3,3′-dichloro-4,4′-diaminodiphenylmethane (MBOCA), trimethylene glycol di-p-aminobenzoate (Polacure 740M). Aliphatic aminic chain extenders can likewise be used or used in part. Propane-1,3-diol, butane-1,4-diol, butane-2,3-diol, pentane-1,5-diol, hexane-1,6-diol and HQEE (hydroquinone di(β-hydroxyethyl) ether), and also water. Very particular preference is given to using butane-1,4-diol for bulk cast elastomers and water for cellular cast elastomers.

An overview of polyurethanes and their properties and uses is given, for example, in the Kunststoffhandbuch [Plastics Handbook], volume 7, Polyurethane [Polyurethanes], 3rd newly revised edition, volume 193, edited by Prof. Dr. G. W. Becker and Prof. Dr. D. Braun (Carl-Hanser-Verlag, Munich, Vienna).

Preference is given to using NCO-terminated prepolymers having an NCO content of 2% to 15% by weight, very particularly of 2-10% by weight. The room temperature solid diisocyanate is preferably reacted with polyols of functionality 2 to 3, preferably 2, and OH number 28-112 mg KOH/g of substance to give prepolymers. Preference is given to using ester-based polyols. The NCO prepolymers thus prepared are either converted further directly or stored as storage-stable prepolymers in drums, for example, until they are ultimately used. Preference is given to using 1,5-NDI-based prepolymers. The production of the cast elastomers (molded articles) is advantageously conducted at an NCO/OH ratio of 0.7 to 1.30. In the case of cellular elastomers, the amount of the mixture introduced into the mold is typically such that the shaped bodies obtained have the density already described. The starting components are typically introduced into the mold at a temperature of 30 to 110° C. The degrees of densification are between 1.1 and 8, preferably between 2 and 6. The cellular elastomers are appropriately produced by a low-pressure technique or especially the reactive injection molding technique (RIM) in open molds, preferably closed molds.

The reactive injection molding technique is described, for example, by H. Piechota and H. Rohr in “Integral Schaumstoffe” [Integral Foams], Carl Hanser-Verlag, Munich, Vienna 1975; D. J. Prepelka and J. L. Wharton in Journal of Cellular Plastics, March/April 1975, pages 87 to 98 and U. Knipp in Journal of CellularPlastics, March/April 1973, pages 76-84.

Additives such as castor oil or carbodiimides (for example Stabaxols from Rheinchemie as hydrolysis stabilizer, 2,2′,6,6′-tetraisopropyldiphenylcarbodiimide is a known representative) can be added either to the polyol or to the prepolymer. Water, emulsifiers, catalysts and/or auxiliaries and/or additives commonly form the polyol component together with the polyol.

For better demolding, it is customary to provide the molds with external separating agents, for example wax- or silicone-based compounds or aqueous soap solutions. The demolded shaped bodies are typically subjected to subsequent heat treatment at temperatures of 70 to 120° C. for 1 to 48 hours.

Emulsifiers used are, for example, sulfonated fatty acids and further commonly known emulsifiers, for example polyglycol esters of fatty acids, alkylaryl polyglycol ethers, alkoxylates of fatty acids, preferably polyethylene glycol esters, polypropylene glycol esters, polyethylene-polypropylene glycol esters, ethoxylates and/or propoxylates of linoleic acid, linolenic acid, oleic acid, arachidonic acid, more preferably oleic acid ethoxylates. Alternatively, it is also possible to use polysiloxanes. Salts of fatty acids with amines, e.g. diethylammonium oleate, diethanolammonium stearate, diethanolammonium ricinoleate, salts of sulfonic acids, e.g. alkali metal or ammonium salts of dodecylbenzene- or dinaphthylmethanedisulfonic acid, are likewise preferred.

The sulfonated fatty acids can preferably be used as aqueous solutions, for example as a 50% solution. Typical known products are SV and SM additives from Rheinchemie, and WM additive from Rheinchemie as a nonaqueous emulsifier.

The process for producing the cellular PUR cast elastomers is conducted in the presence of water. The water acts both as crosslinker with formation of urea groups and as blowing agent on account of the reaction with isocyanate groups to form carbon dioxide. The amounts of water that can appropriately be used are 0.01% to 5% by weight, preferably 0.3% to 3.0% by weight, based on the weight of the polyol component. The water may be used entirely or partly in the form of the aqueous solutions of the sulfonated fatty acids.

The catalysts may be added individually or else in a blend with one another. These are preferably organometallic compounds such as tin(II) salts of organic carboxylic acids, e.g. tin(II) dioctoate, tin(II) dilaurate, dibutyltin diacetate and dibutyltin dilaurate, and tertiary amines such as tetramethylethylenediamine, N-methylmorpholine, diethylbenzylamine, triethylamine, dimethylcyclohexylamine, diazabicyclooctane, N,N′-dimethylpiperazine, N-methyl-N′-(4-N-dimethylamino)butylpiperazine, N,N,N′,N″,N″-pentamethyldiethylenetriamine or the like. Further useful catalysts include amidines, for example 2,3-dimethyl-3,4,5,6-tetrahydropyrimidine, tris(dialkylaminoalkyl)-s-hexahydrotriazines, especially tris(N,N-dimethylaminopropyl)-s-hexahydrotriazine, tetraalkylammonium hydroxides, for example tetramethylammonium hydroxide, alkali metal hydroxides, for example sodium hydroxide, and alkali metal alkoxides, for example sodium methoxide and potassium isopropoxide, and alkali metal salts of long chain fatty acids having 10 to 20 carbon atoms and optionally pendant OH groups. According to the reactivity to be established, the catalysts are employed in amounts of 0.001% to 0.5% by weight, based on the isocyanate component.

The present invention further provides a process for producing an elastomer, in which at least one composition of the invention is cured, optionally while heating, and an elastomer obtained or obtainable by curing, optionally while heating, of the composition of the invention.

The polyurethanes, elastomers or shaped bodies of the invention differ from the products based on monomeric, room temperature solid diisocyanates known from the prior art, preferably those based on naphthalene 1,5-diisocyanate, in that, by virtue of the process of the invention, the contamination of the monomeric, room temperature solid diisocyanate recovered from the residue with these auxiliaries can also be minimized or even entirely avoided.

Such cellular PUR cast elastomers, also referred to as shaped bodies, find use as damping elements in vehicle construction, for example in automobile construction, for example as overload springs, buffers, transverse link bearings, rear axle subframe bearings, stabilizer bearings, longitudinal strut bearings, suspension strut bearings, shock absorber bearings, or bearings for wishbones, and also as an emergency wheel on the rim, which has the effect that the vehicle, for example in the event of tire damage, runs on the cellular elastomer and remains controllable. The bulk cast elastomers can be used for rolls, wheels and drums, squeegees, screens or hydrocyclones.

### EXAMPLE 1

A residue from a distillation of naphthalene 1,5-diisocyanate that has been prepared by phosgenation of naphthalene-1,5-diamine was applied to a Kofler heating bench. The residue still contained about 75% by weight of monomeric naphthalene 1,5-diisocyanate (determined as area % by GPC according to DIN 55672-1:2007-08). The Kofler heating bench was operated at 100 to 250° C. At 250° C. there was rapid embrittlement within 2 minutes, and the residue could be scratched off as a fine powder. The embrittlement also continued subsequently in regions at lower temperatures and, after about 3 hours, regions had also become embrittled at 160° C. and could be scratched off in the form of flakes. Below 130° C., there was no further embrittlement.

### EXAMPLE 2

A residue from a distillation of naphthalene 1,5-diisocyanate that has been prepared by phosgenation of naphthalene-1,5-diamine was distilled under vacuum conditions in a laboratory distillation apparatus. The distillation apparatus was equipped with a torque-measuring stirrer, and the torque was observed in the course of the experiment. The pressure within the distillation apparatus was 2 mbar, and the temperature in the liquid phase at the end of the distillation was about 260° C. After a distillation time of about 7 minutes, there was a brief slight rise in torque up to 10 Ncm. Subsequently, the torque measured dropped again to values of <1 Ncm to form a brittle solid. After 60 minutes, the experiment was ended.

### EXAMPLE 3

A residue from a distillation of tolylene diisocyanate that has been prepared by phosgenation of tolylenediamine in the gas phase was distilled under vacuum conditions in a laboratory distillation apparatus. The distillation apparatus was equipped with a torque-measuring stirrer, and the torque was observed in the course of the experiment. The pressure within the distillation apparatus was 100 mbar, and the temperature in the liquid phase at the end of the distillation was about 260° C. After a distillation time of about 15 minutes, there was a constant rise in torque. After 90 minutes, the experiment was ended. The torque had risen to 55 Ncm, and a highly viscous, thick mass remained in the liquid phase. No embrittlement was observed.

1. A process for recovering monomeric, room temperature solid diisocyanates from a distillation residue, comprising the following steps:
(i) providing at least one residue containing a room temperature solid diisocyanate, and
(ii) separating the residue in at least one selected from the group consisting of a kneader-drier, a paddle drier and a drum drier in the presence of less than 2% by weight of bitumen, based on the mass of the residue provided in step (i), into a gaseous portion containing monomeric, room temperature solid diisocyanate, and a brittle residue depleted of room temperature solid diisocyanate.

2. The process as claimed in claim 1, wherein the kneader-drier, paddle drier or drum drier is designed without a cooling zone.

3. The process as claimed in claim 1, wherein the residue is separated in step (ii) in a drum drier.

4. The process as claimed in claim 1, wherein the separation in step (ii) is effected in the presence of ≤1% by weight of bitumen.

5. The process as claimed in claim 1, wherein the temperature in step (ii) is ≥130° C. to ≤270° C.

6. The process as claimed in claim 1, wherein the pressure in step (ii) is ≥1 mbar to ≤1020 mbar.

7. The process as claimed in claim 1, wherein the room temperature solid diisocyanate is selected from the group consisting of naphthalene 1,5-diisocyanate, naphthalene 1,8-diisocyanate, and phenylene 1,4-diisocyanate.

8. The process as claimed in claim 1, wherein the residue in the separation in step (ii) has an average dwell time in the at least one kneader-drier, paddle drier or drum drier of ≥0.5 minute to ≤4 hours minutes.

9. The process as claimed in claim 1, wherein the brittle residue obtained in step (ii) is discharged semicontinuously.

10. The process as claimed in claim 1, wherein the brittle residue obtained in step (ii) contains ≤5% by weight, of monomeric, room temperature solid diisocyanate based on the total mass of the residue.

11. The process as claimed in claim 1, wherein the residue containing room temperature solid diisocyanates that is provided in step (i) comes from the distillation of a diisocyanate prepared by phosgenating the corresponding diamines.

12. The monomeric, room temperature solid diisocyanate obtained by the process as claimed in claim 1.

13. In a process for minimizing the use of auxiliaries in the embrittlement of a residue containing room temperature solid diisocyanates, the improvement comprising including one of a kneader-drier, a paddle drier and a drum drier.

14. A composition comprising the at least one monomeric, room temperature solid diisocyanate as claimed in claim 12 and at least one NCO-reactive compound.

15. An elastomer obtained by curing, optionally while heating, the composition as claimed in claim 14.

16. The process as claimed in claim 1, wherein the separation in step (ii) is effected in the absence of bitumen.

17. The composition as claimed in claim 14, wherein the at least one NCO-reactive compound is a polyester polyol.
